(19) United States
(12) Patent Application Publication
(10) Pub. No.: US 2010/0099950 A1
(43) Pub. Date: Apr. 22, 2010

(54) ENDOSCOPE SYSTEM

(75) Inventor: Yasuhiro Kamihara, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/525,664

(22) PCT Filed: Feb. 4, 2008
(86) PCT No.: PCT/JP2008/051731

§ 371 Date: Aug. 3, 2009

(30) Foreign Application Priority Data
Feb. 5, 2007 (JP) 2007-025783

(51) Int. Cl. A61B 1/045

(52) U.S. Cl. 600/118

(57) ABSTRACT

Even in the event of movement by the operator, vibrations received from outside, changes in orientation etc., the spectral characteristics can be controlled with high precision, thus enabling acquisition of a desired observation image. Provided is an endoscope system including two optical substrates (13a, 13b) that oppose each other with a distance therebetween; an actuator (13c) that varies the distance between the two optical substrates (13a, 13b) according to an input driving signal; sensors (16) that are fixed to the two optical substrates (13a, 13b) and that detect the distance between the optical substrates (13a, 13b); an electrical circuit (17) that is disposed in the vicinity of the optical substrates (13a, 13b), that has outputs from the sensors (16) input thereto, and that includes an active element and outputs an electrical signal corresponding to the outputs from the sensors (16); and a securing portion (25) that secures electrical wires (24) connecting the electrical circuit (17) and each of the sensors (16), at any position between the electrical circuit (17) and the sensors (16).

### TECHNICAL FIELD

The present invention relates to an endoscope system.

### BACKGROUND ART

There are known technologies in which an etalon device that can vary the distance between a plurality of optical substrates with driving means formed of piezoelectric devices is disposed in at least one of an image-acquisition optical system and an illumination optical system at the tip of an endoscope to vary the wavelength characteristics of observed light or illumination light (for example, see Patent Document 1).

By using this technology disclosed in Patent Document 1, it is possible to obtain spectral information about a living organism etc. In the etalon device disclosed in Patent Document 1, driving means formed of piezoelectric devices are provided between two or more optical substrates for varying the distance between these optical substrates.

Patent Document 1: the Publication of Japanese Patent No. 2802061

### DISCLOSURE OF INVENTION

The present invention provides an endoscope system that can control the spectral characteristics with high precision to obtain a desired observation image, even in the event of movement by the operator, vibrations received from outside, changes in orientation, or the like.

One aspect of the present invention is an endoscope system for obtaining an image of an image-acquisition target inside a body cavity of a living organism, including an insertion portion that is inserted inside the body cavity; two optical substrates that oppose each other with a distance therebetween; an actuator that varies the distance between the two optical substrates according to an input driving signal; sensors that are fixed to the two optical substrates and that detect the distance between the optical substrates; an electrical circuit that is disposed in the vicinity of the optical substrates, that has outputs from the sensors input thereto, and that includes an active element and outputs an electrical signal corresponding to the outputs from the sensors; and a securing portion that secures electrical wires connecting the electrical circuit and the sensors, at any position between the electrical circuit and the sensors.

In the above aspect, the electrical circuit may include an amplifier circuit.

In the above aspect, the electrical circuit may include a buffer circuit.

In the above aspect, the sensors may include respective electrodes on the two optical substrates and may detect the distance between the two optical substrates by detecting the electrostatic capacitance between the electrodes.

In the above aspect, the sensors may include a coil provided on one of the two optical substrates and a metal plate provided on the other and may detect the distance between the two optical substrates by detecting the impedance of the coil.

The sensors may include respective electrodes on the two optical substrates, and by detecting the electrostatic capacitance between the electrodes, in the structure for detecting the distance between the two optical substrates, the electrical circuit may convert the electrostatic capacitance generated between the electrodes of the sensors to an electrical signal.

In the above aspect, the securing portion may secure the electrical wires to the optical substrates.

In the above configuration, the securing portion may secure the electrical wires to the optical substrate at a fixed side.

In the above aspect, the securing portion may secure the electrical wires to a base member to which the fixed-side optical substrate is fixed.

In the above aspect, the securing portion may secure the electrical wires to a terminal block that relays the electrical wires from the sensors to the electrical circuit.

In the above aspect, the securing portion may secure the electrical wires with an adhesive.

In the above aspect, the securing portion may secure a plurality of the electrical wires from the sensors to each other.

In the above configuration, the securing portion may secure the electrical wires from the sensors provided on different optical substrates to each other.

In the above configuration, the securing portion may secure the electrical wires from the sensors provided on the same optical substrate to each other.

In the above aspect, a variable spectral device including the optical substrate, the actuator, and the sensors may be disposed at a distal end of the insertion portion.

In the above aspect, a variable spectral device including the optical substrate, the actuator, and the sensors may be disposed at a base end at the opposite end from the distal end of the insertion portion.

The present invention affords an advantage in that it is possible to control spectral characteristics with high precision and to acquire a desired observation image, even in the event of movement by the operator, vibrations received from outside, changes in orientation, or the like.

### BRIEF DESCRIPTION OF DRAWINGS

FIG. 1 is a block diagram showing the overall configuration of an endoscope system according to a first embodiment of the present invention.

FIG. 2 is a longitudinal sectional view schematically showing an image-acquisition unit of the endoscope system in FIG. 1.

FIG. 3 is a block diagram for explaining the internal wiring connections of the image-acquisition unit of the endoscope system in FIG. 1.

FIG. 4 is a diagram showing transmittance characteristics of a variable spectral device constituting the image-acquisition unit in FIG. 2.

FIG. 5 is a timing chart for explaining the operation of the endoscope system in FIG. 1.

FIG. 6 is a diagram showing an electrical circuit for amplifying a sensor signal of the variable spectral device provided in the image-acquisition unit in FIG. 2.

FIG. 7 is a longitudinal section view showing a first modification of the image-acquisition unit in FIG. 2.

FIG. 8 is a longitudinal sectional view showing a second modification of the image-acquisition unit in FIG. 2.

FIG. 9 is a longitudinal sectional view showing a third modification of the image-acquisition unit in FIG. 2.

FIG. 10 is a longitudinal sectional view showing a fourth modification of the image-acquisition unit in FIG. 2.

FIG. 11 is a longitudinal sectional view showing a fifth modification of the image-acquisition unit in FIG. 2.

FIG. 12 is a longitudinal sectional view showing a sixth modification of the image-acquisition unit in FIG. 2.

FIG. 13 is a longitudinal sectional view showing a seventh modification of the image-acquisition unit in FIG. 2.

FIG. 14 is a schematic diagram showing an example of a fiber-type endoscope system in which an image-acquisition unit is disposed at the base end of an insertion portion.

FIG. 15 is a longitudinal section view schematically showing a light source unit in an endoscope system according to a second embodiment of the present invention.

### EXPLANATION OF REFERENCE SIGNS

A: image-acquisition target
1: endoscope system
2: insertion portion
13 variable spectral device
13a, 13b: optical substrate
13c: actuator
15: frame member (base member)
16: sensor
16a, 16b: sensor electrode (electrode)
17: electrical circuit
22: op-amp (amplifier circuit)
24: electrical wire
25: adhesive (securing portion)

### BEST MODE FOR CARRYING OUT THE INVENTION

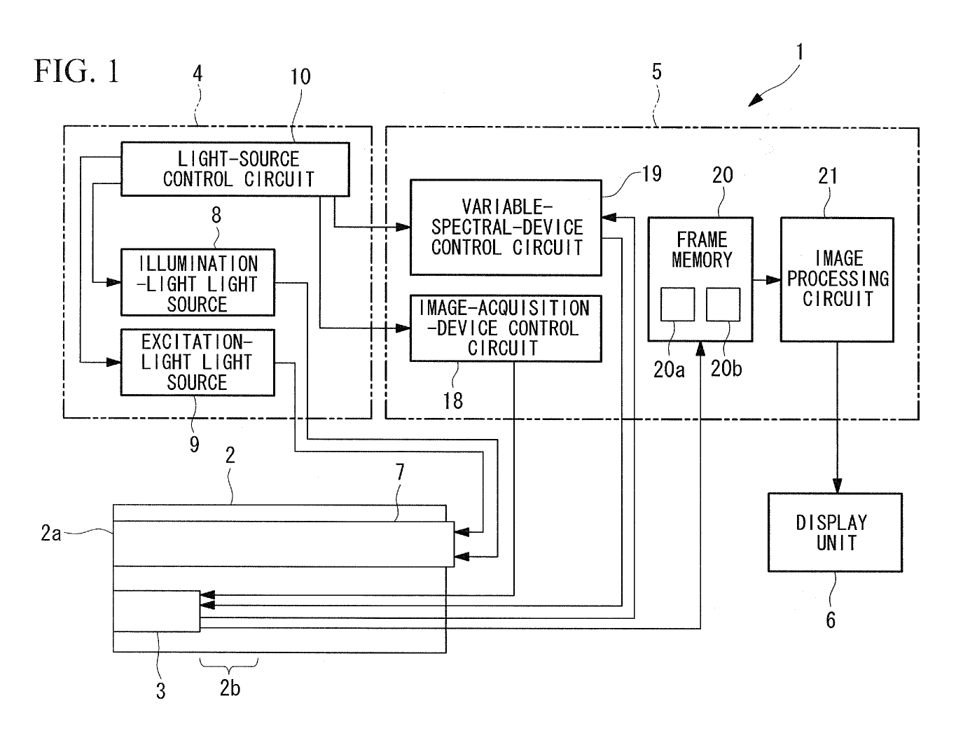

An endoscope system 1 according to a first embodiment of the present invention will be described below with reference to FIGS. 1 to 6.

As shown in FIG. 1, the endoscope system 1 includes an insertion portion 2 that is inserted inside the body cavity of a living organism, an image-acquisition unit 3 disposed inside the insertion portion 2, a light-source unit 4 that emits a plurality of kinds of light, a control unit 5 that controls the image-acquisition unit 3 and the light-source unit 4, and a display unit 6 that displays an image acquired by the image-acquisition unit 3.

The insertion portion 2 has extremely narrow outer dimensions allowing it to be inserted into the body cavity of the living organism, and the image-acquisition unit 3 and a light guide 7 that conveys light from the light source unit 4 to a tip 2a are provided in the interior thereof.

The light-source unit 4 illuminates an observation target A inside the body cavity and includes an illumination-light light source 8 that emits illumination light that is reflected at the observation target A to obtain returning reflected light, an excitation-light light source 9 that emits excitation light that is radiated onto the observation target in the body cavity to excite a fluorescent substance present in the observation target and generate fluorescence, and a light-source control circuit 10 that controls the light sources 8 and 9.

The illumination-light light source 8 is, for example, a combination of a xenon lamp and a bandpass filter, which are not illustrated in the drawing, and the 50% transmission band of the bandpass filter is 430 to 460 nm. In other words, the light source 8 generates illumination light in the wavelength band of 430 to 460 nm.

The excitation-light light source 9 is a semiconductor laser that emits excitation light with a peak wavelength of, for example, 660±5 nm. Excitation light of this wavelength can excite a fluorescent agent such as Cy5.5 (manufactured formerly by Amersham, currently GE Health Care Inc.) or ALEXAFLUOR700 (manufactured by Molecular Probes).

The light-source control circuit 10 is configured to alternately turn on and off the illumination-light light source 8 and the excitation-light light source 9 at a prescribed timing according to a timing chart described later.

The image-acquisition unit 3 is disposed at a distal end of the insertion portion 2.

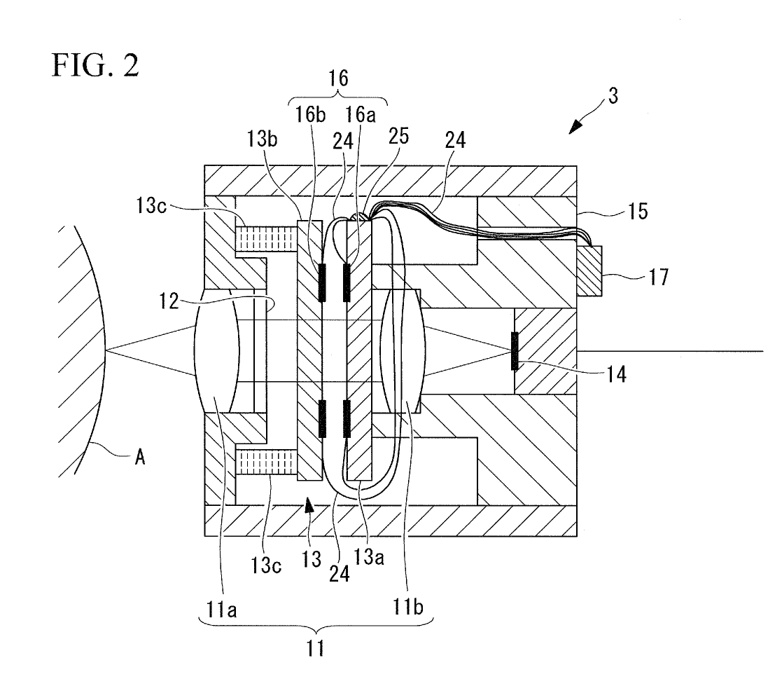

As shown in FIG. 2, the image-acquisition unit 3 includes an image-acquisition optical system 11 including lenses 11a and 11b that collect light incident from an observation target A, an excitation-light cut filter 12 that blocks excitation light incident from the observation target A, a variable spectral device 13 whose spectral characteristics are varied by the operation of a control unit 5, an image-acquisition device 14 that captures and converts the light collected by the image-acquisition optical system 11 into an electrical signal, and a frame member 15 that supports these components.

The variable spectral device 13 is an etalon-type optical filter provided with two circular plate-shaped optical substrates 13a and 13b disposed in parallel with a distance therebetween and having reflective films provided on opposing surfaces, and actuators 13c that vary the distance between the optical substrates 13a and 13b. The optical substrate 13a is directly fixed to the frame member 15, and the optical substrate 13b is attached to the frame member 15 with the actuator 13c interposed therebetween.

The actuators 13c are multilayer piezoelectric devices which are provided at four locations at equal intervals in the circumferential direction around the edge of the optical substrate 13b.

This variable spectral device 13 can vary the distance between the optical substrates 13a and 13b by the operation of the actuators 13c, thereby enabling it to change the wavelength band of light transmitted in the axial direction.

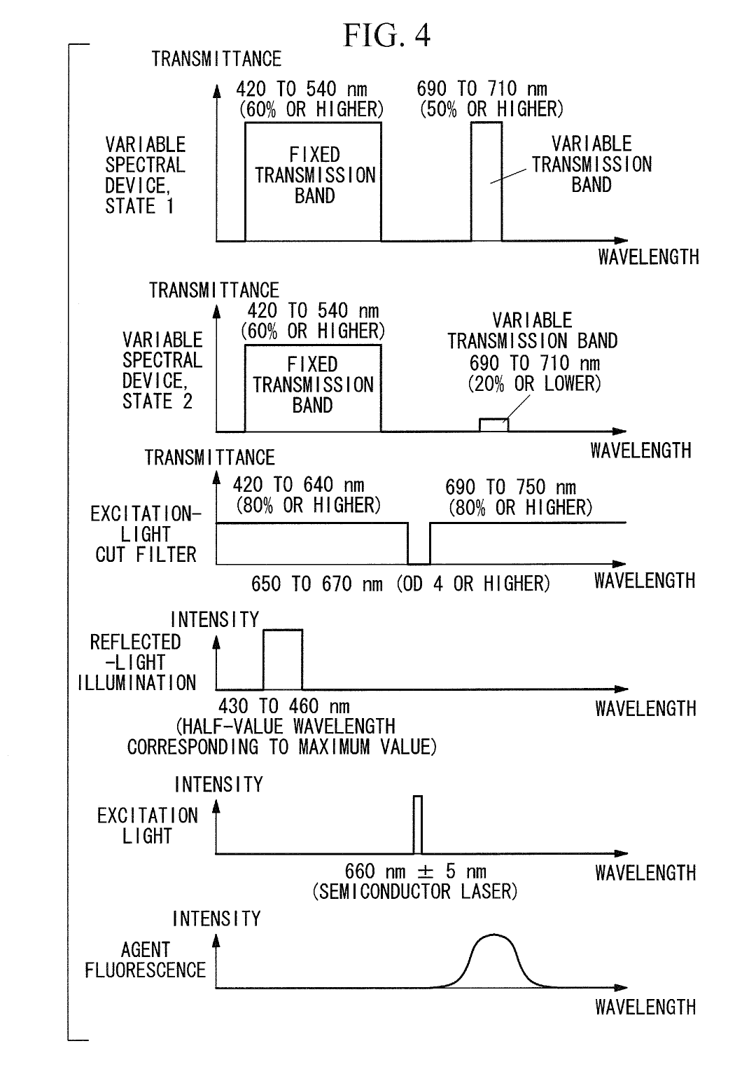

More specifically, as shown in FIG. 4, the variable spectral device 13 has a transmittance-wavelength characteristic with two transmission bands, namely, one fixed transmission band and one variable transmission band. The fixed transmission band always transmits incident light regardless of the state of the variable spectral device 13. In the variable transmission band, the transmittance characteristic varies according to the state of the variable spectral device 13.

The two optical substrates 13a and 13b constituting the variable spectral device 13 are provided with sensors 16 for detecting the distance between the optical substrates 13a and 13b. The sensors 16 are of the electrostatic capacitance type and include a plurality of sensor electrodes 16a and 16b respectively provided at mutually opposing positions in the outer peripheral portion outside the optically effective diameter of the optical substrates 13a and 13b. The sensor electrodes 16a and 16b are disposed at four positions in the outer peripheral portion of the optical substrates 13a and 13b at equal intervals in the circumferential direction. Metal films can be used as the sensor electrodes 16a and 16b.

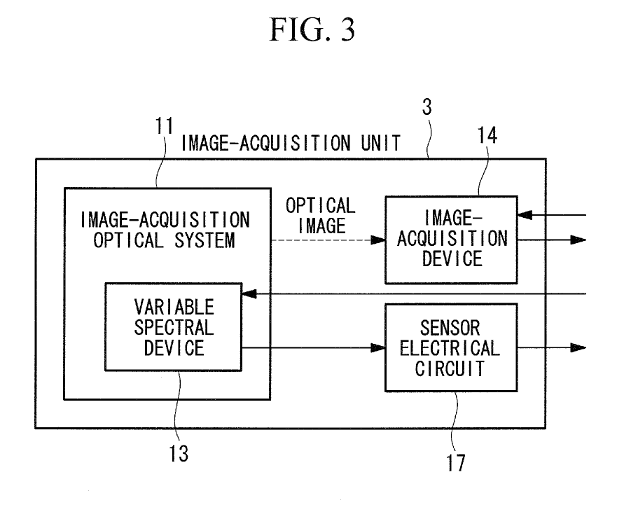

Electrostatic capacitance type devices utilize a characteristic whereby the electrostatic capacitance between the sensor electrodes 16a and 16b is inversely proportional to the distance between the surfaces. An electrical circuit 17 is connected to the sensor electrodes 16a and 16b via electrical wires 24. In other words, as shown in FIG. 3, the variable spectral device 13 of the image-acquisition unit 3 is electrically connected to the electrical circuit 17 for the sensors 16, and the output of the electrical circuit 17 is connected to a variable-spectral-device control circuit 19 shown in FIG. 1.

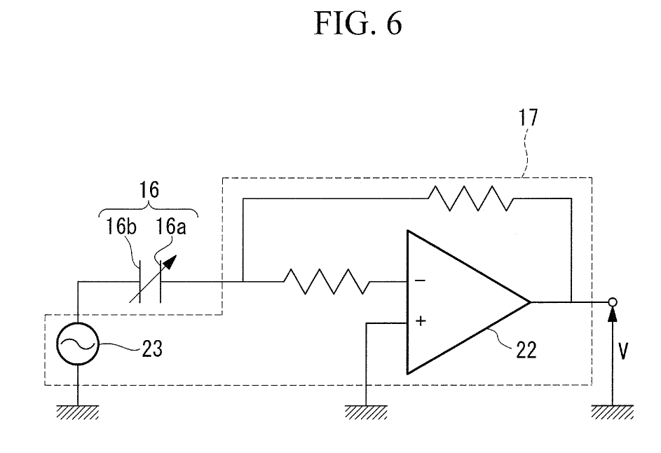

As shown in FIG. 6, for example, the electrical circuit 17 supplies AC current to the sensor electrodes 16a and 16b, converts the electrostatic capacitance between the sensor electrodes 16a and 16b, which is determined by the distance between the optical substrates 13a and 13b, to a voltage signal, amplifies the signal, and outputs voltage V. In FIG. 6, reference sign 22 is an op-amp, which is an active element, and reference sign 23 is an AC power supply. The electrical circuit 17 is fixed to the frame member 15.

In this embodiment, the electrical wire 24 leading from each sensor electrode 16a and 16b is secured with an adhesive 25 to the fixed-side optical substrate 13a between the sensor electrodes 16a and 16b and the electrical circuit 17.

The amount of adjustment of the distance between the optical substrates 13a and 13b by the actuators 13c is minute, and the variation in the electrostatic capacitance between the sensor electrodes 16a and 16b is also correspondingly very small. Thus, the variation in the electrostatic capacitance (parasitic capacitance) formed between the electrical wires 24 leading from the sensor electrodes 16a and 16b also cannot be ignored.

According to this embodiment, because the electrical wires 24 leading from the sensor electrodes 16a and 16b are secured to the optical substrate 13a by the adhesive 25, displacement of the electrical wires is limited to a displacement associated with the displacement of the optical substrate 13b. Therefore, even if the insertion portion 2 is moved by the operator, vibrations are received from the body cavity, or the orientation of the patient changes, the electrical wires 24 between the sensor electrodes 16a and 16b and the electrical circuit 17 are not displaced.

In fluorescence observation, because the obtained fluorescence intensity is generally very weak, the transmission efficiency of the optical system is extremely important. The etalon-type variable spectral device 13 has a high transmittance when the reflective films are parallel; however, if there is an error in adjusting the degree of parallelism, the transmittance abruptly decreases. Therefore, as the variable spectral device 13 used in the image-acquisition unit 3 for fluorescence observation, in order to adjust the tilt error of the two optical substrates 13a and 13b when the distance therebetween changes, it is preferable to provide a plurality of sensors 16 to have multiple degrees of freedom for driving.

Based on the signals from the sensor electrodes 16a and 16b, it is possible to improve the precision in controlling the transmittance characteristic by implementing feedback control of the driving signals to the actuators 13c.

In this embodiment, the variable spectral device 13 has a variable transmission band in a wavelength band (for example, 690 to 710 nm) containing the wavelengths of fluorescence (agent fluorescence) generated by exciting the fluorescent agent with the excitation light. Thus, the variable spectral device 13 changes to two states according to a control signal from the control unit 5.

The first state is a state in which the transmittance in the variable transmission band is increased to 50% or more, allowing transmission of the agent fluorescence. The second state is a state in which the transmittance in the variable transmission band is reduced to 20% or less to block the agent fluorescence.

The second state may block agent fluorescence by changing the wavelength band of the variable transmission band from that in the first state.

The fixed transmission band is located in a range of, for example, 420 to 540 nm and is designed to have an average transmittance of 60% or more.

Also, the fixed transmission band is located in the wavelength band including the wavelengths of reflected light of the illumination light so that it is possible to transmit the reflected light towards the image-acquisition device 14 in either the first or the second state.

The excitation-light cut filter 12 has a transmittance of 80% or more in a wavelength band from 420 to 640 nm, an OD value of 4 or more (=transmittance of 1×10⁻⁴ or less) in a wavelength band of 650 to 670 nm, and a transmittance of 80% or more in a wavelength band of 690 to 750 nm.

As shown in FIG. 1, the control unit 5 includes an image-acquisition-device control circuit 18 for drive control of the image-acquisition device 14, a variable-spectral-device control circuit 19 for drive control of the variable spectral device 13, a frame memory 20 that stores image information acquired by the image-acquisition device 14, and an image processing circuit 21 that processes the image information stored in the frame memory 20 and outputs it to the display unit 6.

The image-acquisition-device control circuit 18 and the variable-spectral-device control circuit 19 are connected to the light-source control circuit 10 and are configured to drive control the variable spectral device 13 and the image-acquisition device 14 in synchronization with the switching of the illumination-light light source 8 and the excitation-light light source 9 by the light-source control circuit 10.

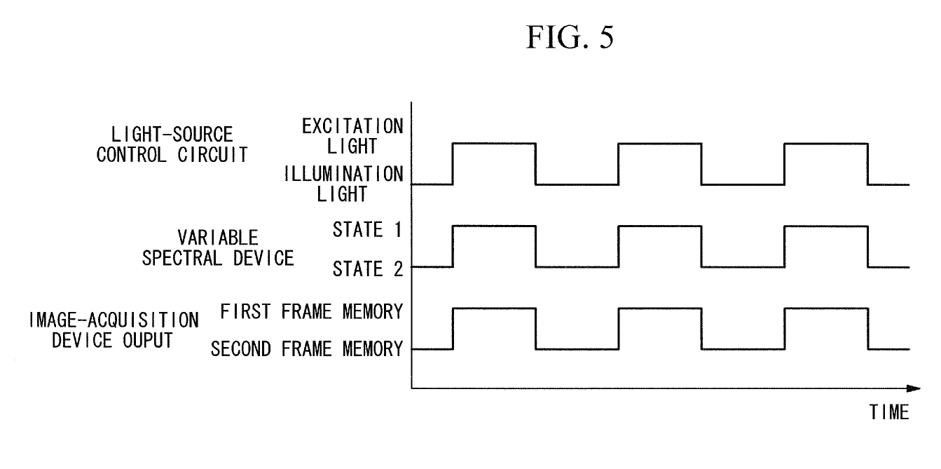

Specifically, as shown in the timing chart in FIG. 5, when the excitation light is emitted from the excitation-light light source 9 by operating the light-source control circuit 10, the variable-spectral-device control circuit 19 sets the variable spectral device 13 to the first state, and the image-acquisition-device control circuit 18 outputs the image information output from the image-acquisition device 14 to a first frame memory 20a. When the illumination light is emitted from the illumination-light light source 8, the variable-spectral-device control circuit 19 sets the variable spectral device 13 to the second state, and the image-acquisition-device control circuit 18 outputs the image information output from the image-acquisition device 14 to a second frame memory 20b.

For example, the image processing circuit 21 receives from the first frame memory 20a the fluorescence image information obtained by radiating the excitation light and outputs it on a first channel of the display unit 6, and receives from the second frame memory 20b the reflected-light image information obtained by radiating the illumination light and outputs it on a second channel of the display unit 6.

The operation of the thus-configured endoscope system 1 according to this embodiment will be described below.

To acquire an image of the image-acquisition target A inside the body cavity of a living organism using the endoscope system 1 according to this embodiment, a fluorescent agent is injected into the body, the insertion portion 2 is inserted inside the body cavity, and a tip 2a thereof is placed opposite the image-acquisition target A inside the body cavity. In this state, the light source unit 4 and the control unit 5 are activated, and by operating the light-source control circuit 10, the illumination-light light source 8 and the excitation-light light source 9 are operated alternately to generate the illumination light and the excitation light, respectively.

The excitation light and the illumination light generated in the light source unit 4 are conveyed to the tip 2a of the insertion portion 2 via the light guide 7 and are radiated from the tip 2a of the insertion portion 2 towards the image-acquisition target A.

When the excitation light is radiated onto the image-acquisition target A, the fluorescent agent permeating the image-acquisition target A is excited, and fluorescence is emitted. The fluorescence emitted from the image-acquisition target A is collected by the image-acquisition optical system 11 of the image-acquisition unit 3, is transmitted through the excitation-light cut filter 12, and is incident on the variable spectral device 13.

Because the variable spectral device 13 is switched to the first state in synchronization with the operation of the excitation-light light source 9 by operating the variable-spectral-device control circuit 19, the transmittance of the fluorescence is increased, thus enabling transmission of the incident fluorescence. In this case, part of the excitation light radiating the image-acquisition target A is reflected at the image-acquisition target A and enters the image-acquisition unit 3 together with the fluorescence; however, because the image-acquisition unit 3 is provided with the excitation-light cut filter 12, the excitation light is blocked and is thus prevented from being incident on the image-acquisition device 14.

Thus, the fluorescence transmitted through the variable spectral device 13 is incident on the image-acquisition device 14, and fluorescence image information is obtained. The obtained fluorescence image information is stored in the first frame memory 20a, is output on the first channel of the display unit 6 by the image processing circuit 21, and is displayed by the display unit 6.

On the other hand, when the illumination light is radiated onto the image-acquisition target A, the illumination light is reflected at the surface of the image-acquisition target A, is collected by the image-acquisition optical system 11, is transmitted through the excitation-light cut filter 12, and is incident on the variable spectral device 13. Because the wavelength band of the reflected illumination light is located in the fixed transmission band of the variable spectral device 13, all of the reflected light incident on the variable spectral device 13 is transmitted through the variable spectral device 13.

Then, the reflected light transmitted through the variable spectral device 13 is incident on the image-acquisition device 14, and reflected-light image information is obtained. The obtained reflected-light image information is stored in the second frame memory 20b, is output on the second channel of the display unit 6 by the image processing circuit 21, and is displayed by the display unit 6.

In this case, because the variable spectral device 13 is switched to the second state in synchronization with the operation of the illumination-light light source 8 by the operation of the variable-spectral-device control circuit 19, the transmittance of the fluorescence is decreased, and even though the fluorescence is incident, the fluorescence is blocked. Accordingly, only the reflected light is captured by the image-acquisition device 14.

In this way, with the endoscope system 1 according to this embodiment, a fluorescence image and a reflected-light image can be provided to the operator.

In this case, with the endoscope system 1 according to this embodiment, because the variable spectral device 13 is provided with the sensors 16, when switching between the first state and the second state, the distance between the two optical substrates 13a and 13b is detected by the sensors 16, and the voltage signal applied to the actuators 13c is feedback controlled. Accordingly, the distance between the optical substrates 13a and 13b can be precisely controlled, light in a desired wavelength band can be spectrally separated with high precision, and a clear fluorescence image and reflected-light image can be obtained.

Furthermore, in this embodiment, the electrical signal indicating the electrostatic capacitance between the sensor electrodes 16a and 16b, which is output from the sensor electrodes 16a and 16b, is amplified by the electrical circuit 17 fixed to the optical substrate 13b of the variable spectral device 13 and the output impedance is reduced; then, it is conveyed inside the insertion portion 2 and is sent from the base end of the insertion portion 2 to the variable-spectral-device control circuit 19 outside the body. Therefore, it is possible to reduce the intrusion of noise in the electrical signal detected by the sensors 16, enabling high-precision detection of the distance between the optical substrates 13a and 13b, which in turn is advantageous in that the spectral characteristics of the variable spectral device 13 can be controlled with high precision.

In this embodiment, the electrical wires 24 leading from the sensor electrodes 16a and 16b are secured by the adhesive 25 to the fixed-side optical substrate 13a at an intermediate position on the way to being connected to the electrical circuit 17; therefore, the electrical wires 24 are prevented from being displaced by a large amount except for cases where they are displaced together with the displacement of the optical substrate 13b. In other words, parasitic capacitance formed by the electrical wires 24 can be prevented from varying independently of the relative displacement of the optical substrates 13a and 13b by the actuators 13c.

Therefore, even if the insertion portion 2 is moved by the operator, or if there are vibrations received from the body cavity or a change in orientation of the patient, the electrical wires 24 do not move, and hence, it is possible to more reliably prevent variation in the distance between the optical substrates 13a and 13b detected by the sensors 16.

In particular, in endoscope examination, advancing/retracting movement, angle movement, and so forth are necessary during observation, and in addition to causing shifting of the variable spectral device 13 itself, at least a portion of the insertion portion 2 often contacts the observation target, which exhibits pulsation etc.; and because vibrations are easily received from the observation target, it is advantageous to use a configuration like that in this embodiment.

In the endoscope system 1 according to this embodiment, the following various modifications and adjustments are possible.

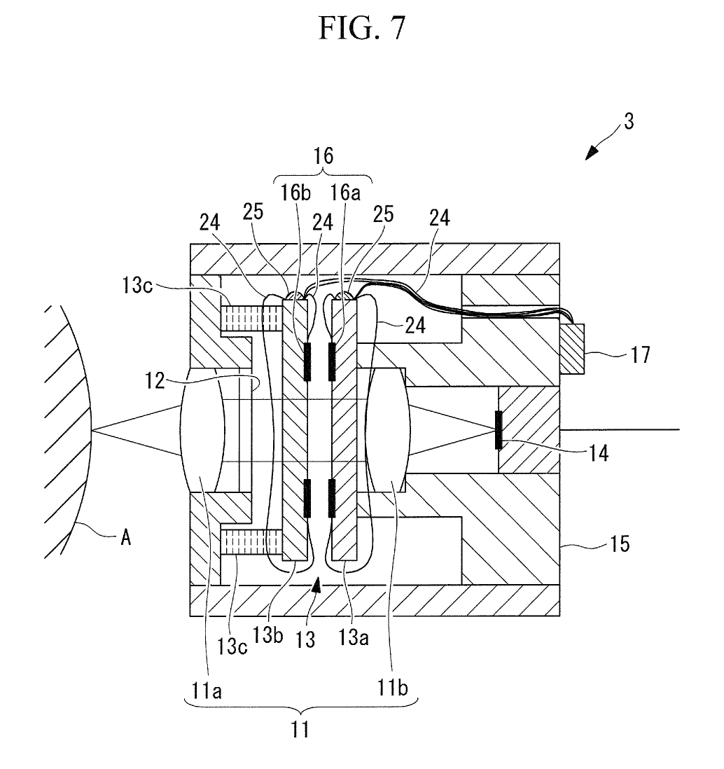

In this embodiment, the electrical wires 24 leading from the sensor electrodes 16a and 16b provided on the respective optical substrates 13a and 13b are secured together to the fixed-side optical substrate 13a by the adhesive 25. Instead of this, as shown in FIG. 7, the electrical wires 24 leading from the sensor electrodes 16a and 16b provided on the respective optical substrates 13a and 13b may be secured by the adhesive 25 to the optical substrates 13a and 13b provided with the respective sensor electrodes 16a and 16b.

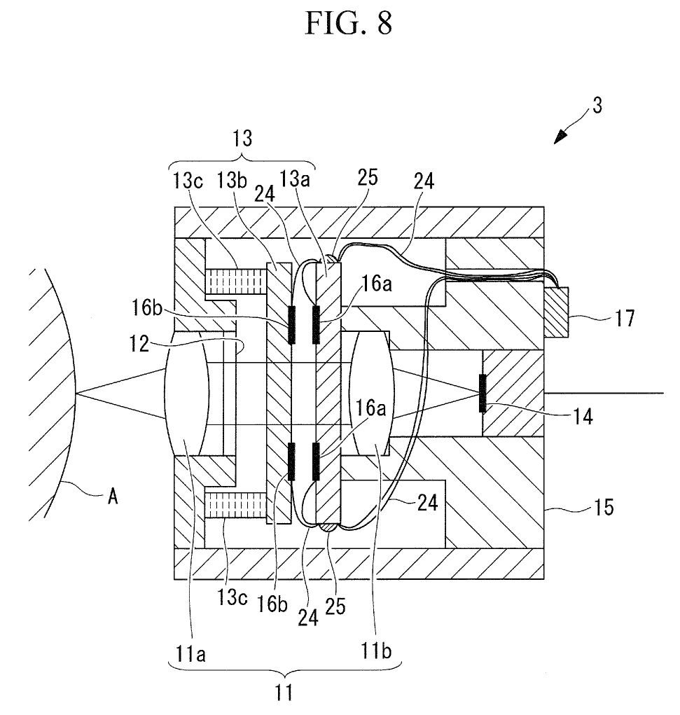

Additionally, as shown in FIG. 8, for each of the plurality of sensor electrodes 16a and 16b provided on the respective optical substrates 13a and 13b at intervals in the circumferential direction, the electrical wires 24 may be secured by the adhesive 25 to the fixed-side optical substrate 13a.

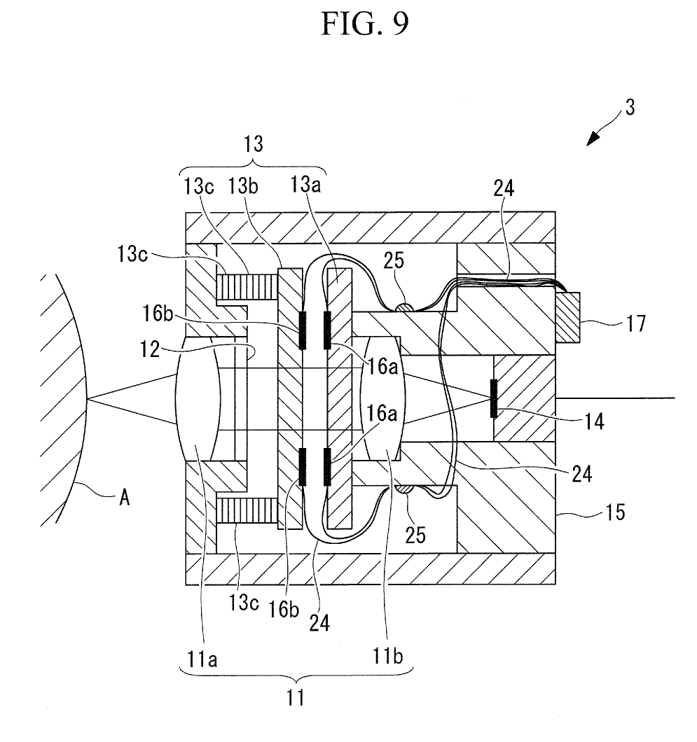

As shown in FIG. 9, the electrical wires 24 may be secured by the adhesive 25 to the frame member 15 to which the fixed-side optical substrate 13a is fixed.

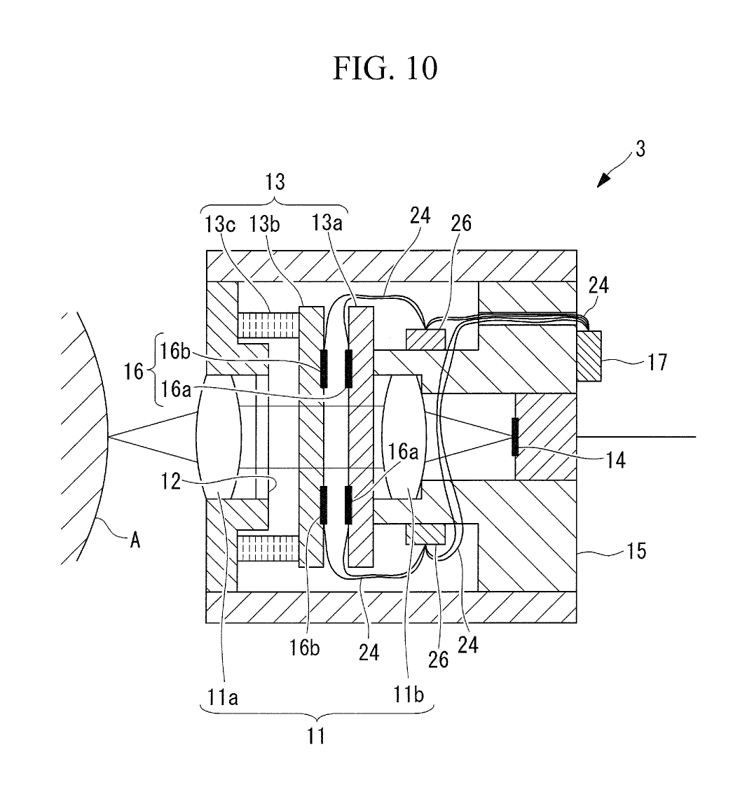

As shown in FIG. 10, the electrical wires 24 may be relayed to terminal blocks 26, which are fixed to the frame member 15 to which the fixed-side optical substrate 13a is fixed.

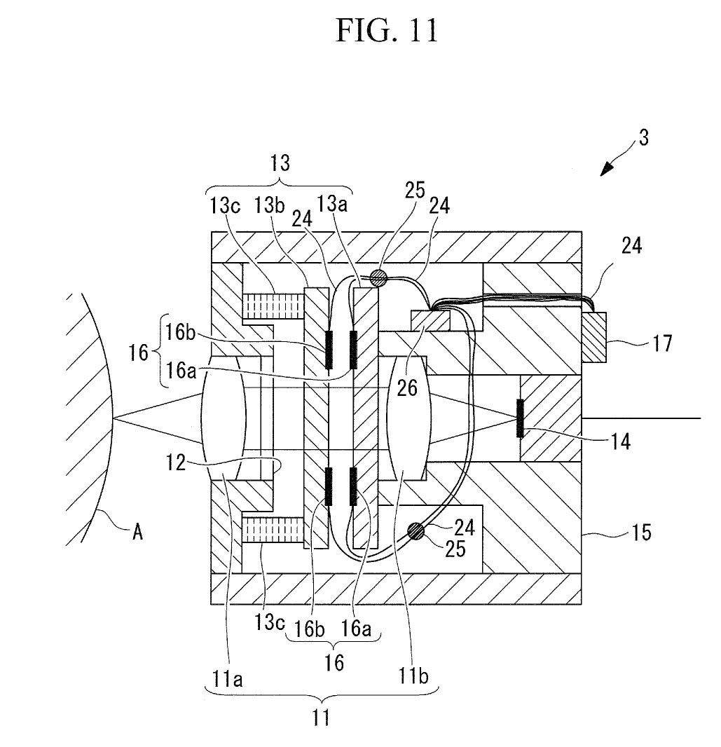

As shown in FIG. 11, the plurality of electrical wires 24 from the sensor electrodes 16a and 16b may be secured together by the adhesive 25 before being secured by the terminal blocks 26.

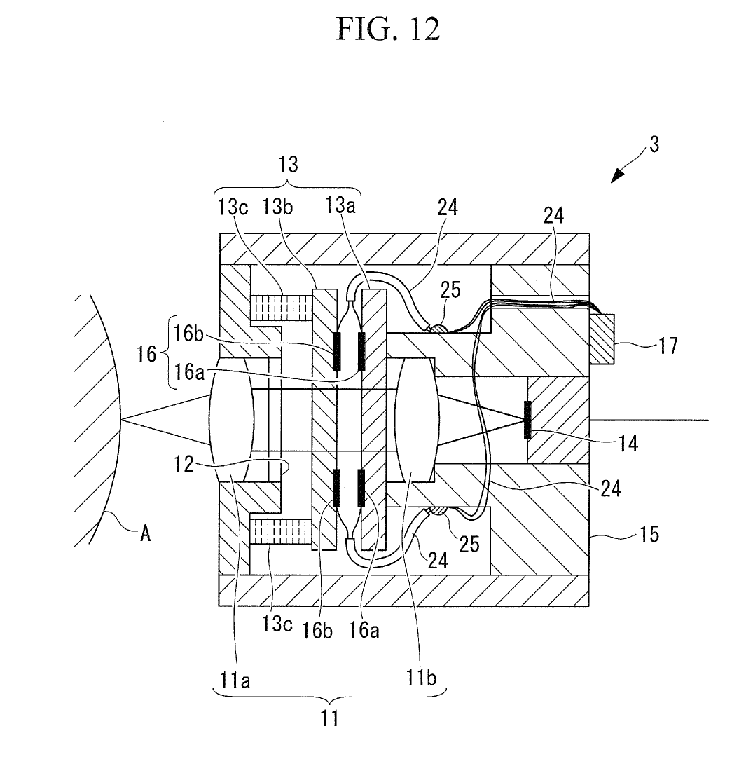

As shown in FIG. 12, feeder lines, twisted-pair lines, or flat cables having insulating coating may be used as the electrical wires 24. By doing so, it is also possible secure the electrical wires 24 from the sensor electrodes 16a and 16b together.

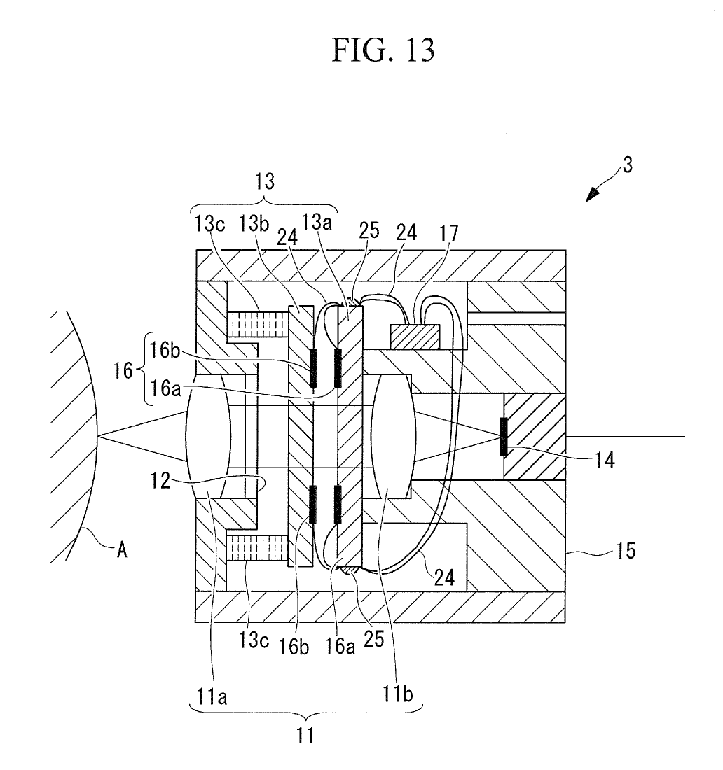

As shown in FIG. 13, the electrical wires 24 may be secured to the fixed-side optical substrate 13a by adhesive 25, at an intermediate position from the sensor 16a and 16b to the electrical circuit 17 disposed in the vicinity of the fixed-side optical substrate 13a.

In the endoscope system 1 according to this embodiment, a description has been given of a case where the reflected-light image and the agent-fluorescence image are acquired in a switching manner; instead of this, however, it is possible to apply the present invention to another observation cases where only the reflected-light image is to be acquired, where an autofluorescence image and an agent-fluorescence image are to be acquired in a switching manner, where an autofluorescence image and a reflected-light image are to be acquired in a switching manner, or the like.

Although a circuit that converts an electrostatic capacitance value to a voltage value is used as the electrical circuit 17, a circuit that converts it to an electrical current value may be used instead.

The sensors 16 used in this embodiment are of the electrostatic capacitance type; however, another type, for example, an eddy current type, may be used instead. The eddy-current type sensor mentioned here is one that generates an eddy current in a target object by means of a high-frequency magnetic field produced by a resonant circuit formed of a coil and a capacitor and that uses a change in magnetic field due to this eddy current to measure displacement. Because the intensity of the magnetic field, which is determined by the distance between the coil and the target object, is detected as a change in impedance of the coil, it is formed by disposing a metal plate on one optical substrate 13a (13b) and disposing the coil at an opposing position on the other optical substrate 13b (13a).

Although a flexible scope in which the insertion portion 2 deforms has been described, instead of this, the present invention may also be applied to a rigid scope or to a capsule endoscope. In addition, the observation target A is not limited to a living organism; the present invention can also be applied to an industrial endoscope for which the observation target A is the interior of a pipe, a machine, a structure, or the like.

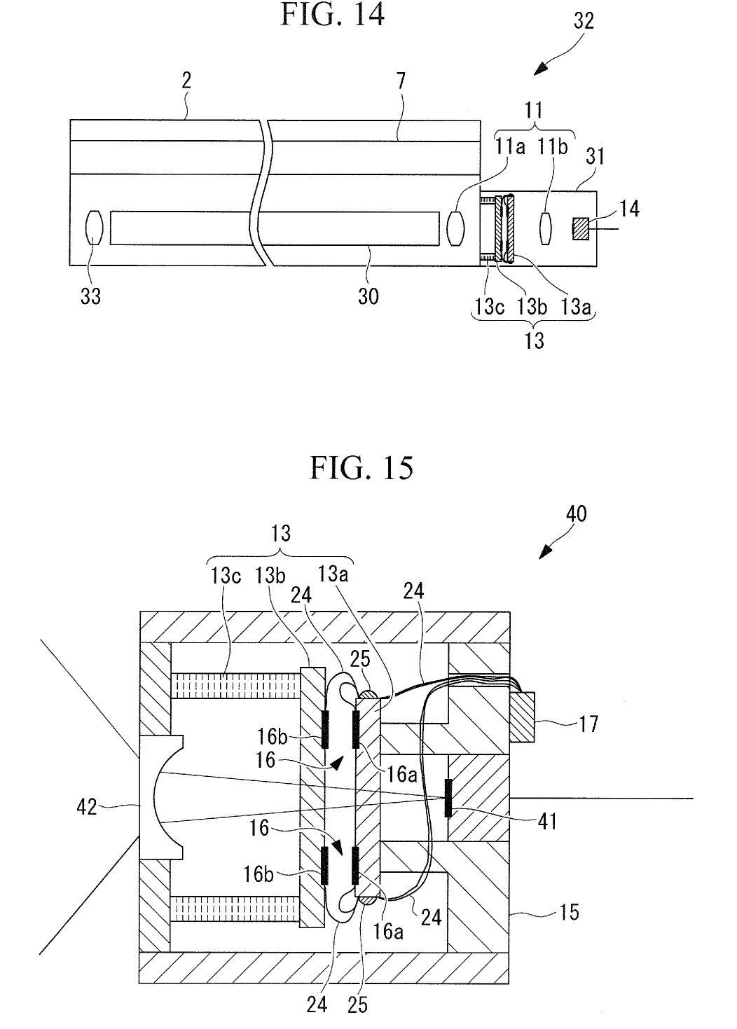

A description has been given, by way of example, of the endoscope system 1 in which the image-acquisition device 14 is disposed at the tip of the insertion portion 2. Instead of this, however, as shown in FIG. 14, the present invention may also be applied to a fiber-type endoscope system 32 in which an image fiber 30 is disposed inside the insertion portion 2, and an image-acquisition unit 31 is provided at the base end of the insertion portion 2. Reference sign 33 in the figure is an objective lens.

Although a circuit that detects electrostatic capacitance in the form of a voltage signal and amplifies the signal is used as the electrical circuit 17, it is not limited thereto; a buffer circuit that has no amplification function may be used. One example of a buffer circuit is a voltage follower circuit. With a buffer circuit, it is also possible to reduce the output impedance of the sensor output, thus enabling improved noise tolerance.

Although a configuration in which the electrical circuit 17 in FIG. 6 is disposed at the tip of the insertion portion is assumed, it is not limited thereto; a configuration in which only the op-amp 22 portion is disposed at the tip of the insertion portion and the AC power supply 23 is disposed outside the insertion portion is also possible.

Next, an endoscope system according to a second embodiment of the present invention will be described below with reference to FIG. 15.

In the description of this embodiment, parts having the same configuration as those in the endoscope system 1 according to the first embodiment described above are assigned the same reference numerals, and a description thereof is omitted.

The endoscope system of this embodiment differs in that, whereas the endoscope system 1 in the first embodiment is provided with the variable spectral device 13 in the image-acquisition unit 3, here the variable spectral device 13 is provided in part of a light source unit 40.

In other words, the light source unit 40 is disposed at the distal end of the insertion portion 2.

The light source unit 40 includes a white LED (photoelectric conversion device) 41 that generates white light, the variable spectral device 13, which is formed of the two optical substrates 13a and 13b and the actuators 13c, a lens 42 that expands the white light emitted from the white LED 41, and a frame member 15 to which these parts are fixed.

The actuators 13c are disposed between the optical substrate 13b and the frame member 15.

The electrical circuit 17, which converts the electrostatic capacitance value detected by the sensors 16 having the sensor electrodes 16a and 16b provided in variable spectral device 13 to a voltage signal and amplifies the signal, is fixed to the frame member 15. Accordingly, it is possible to radiate illumination light in a desired wavelength band onto the living organism A with high transmittance, enabling acquisition of a bright, clear spectral image.

Other than cases where a single white LED 41 is provided, in order to increase the amount of illumination light and improve the light distribution characteristics, a plurality of white LEDs 41 may be disposed in the light source unit 40. Also, a single white LED 41 may be combined with a diffusing plate to increase the light-source area, or a lamp etc. may be used.

It is also possible to use a multiwavelength-excitation semiconductor laser or superluminescent diode.

In the above embodiments, a description has been of the application of a variable-type etalon spectral device to an endoscope system, as well as its operation and advantages; however, this is an example application and the present invention is not limited thereto. For example, an advantage is also afforded when applied to general etalon spectral devices in which the inter-surface distance between a plurality of optical substrates is detected by a sensor and, based on the output from the sensor, the inter-surface distance between these optical substrates is changed with an actuator such as a piezoelectric device (PZT) to control it.

The reason is that, although external vibrations applied to the etalon spectral device itself affect the inter-surface distance detection precision, as explained in the embodiments, the etalon spectral device itself can also be a source of mechanical vibrations. In other words, because the optical substrates constituting the etalon spectral device are also relatively moved with an actuator to change the spectral characteristics, if there is a minute displacement of the optical substrates, it means there will be a source of vibrations inside the spectral device.

Therefore, even with an etalon spectral device built into a stationary apparatus whose positional orientation does not change, it is effective to secure the electrical wires from the sensor in order to reduce the influence of the source of vibrations inside the spectral device to realize high-precision inter-surface distance detection.

Other than endoscopes, application to a microscope, for example, is also possible. Specifically, an etalon spectral device like that described above may be located before an image-acquisition device built into a microscope. When observing a living organism, such as a small animal, or living cells (tissue) with such a microscope, it is assumed that pulsing of the small animal, vibrations of a culture-solution circulating apparatus, or the like will have an influence on the detection precision of the inter-surface distance between the optical substrates. Therefore, the same advantages as in the endoscope described in the embodiments can also be expected in a microscope having such a built-in etalon spectral device described above.

Although embodiments and modifications thereof have been described above, this specification also includes the following inventions.

(1) A variable spectral device comprising:

two optical substrates that oppose each other with a distance therebetween;

an actuator that changes the distance between the two optical substrates according to an input driving signal;

sensors that are fixed to the two optical substrates and that detect the distance between the optical substrates;

an electrical circuit that is disposed in the vicinity of the optical substrates, that has an output from the sensors input thereto, and that contains an active element and outputs an electrical signal corresponding to the output from the sensors; and

a securing portion that secures electrical wires connecting the electrical circuit and each of the sensors at any position between the electrical circuit and the sensors.

(2) A spectral device recited in (1) above, wherein the actuator changes the distance between the optical substrates in a time-division manner, thus obtaining two-dimensional spectral image information in different wavelength bands in a time-division manner.
(3) A spectral apparatus comprising the variable spectral device recited in (1) above.

1. An endoscope system for obtaining an image of an image-acquisition target inside a body cavity of a living organism, comprising:
an insertion portion that is inserted inside the body cavity;
two optical substrates that oppose each other with a distance therebetween;
an actuator that varies the distance between the two optical substrates according to an input driving signal;
sensors that are fixed to the two optical substrates and that detect the distance between the optical substrates;
an electrical circuit that is disposed in the vicinity of the optical substrates, that has outputs from the sensors input thereto, and that includes an active element and outputs an electrical signal corresponding to the outputs from the sensors; and
a securing portion that secures electrical wires connecting the electrical circuit and the sensors, at any position between the electrical circuit and the sensors.

2. An endoscope system according to claim 1, wherein the electrical circuit includes an amplifier circuit.

3. An endoscope system according to claim 1, wherein the electrical circuit includes a buffer circuit.

4. An endoscope system according to claim 1, wherein the sensors comprise respective electrodes on the two optical substrates and detect the distance between the two optical substrates by detecting the electrostatic capacitance between the electrodes.

5. An endoscope system according to claim 4, wherein the electrical circuit converts the electrostatic capacitance generated between the electrodes of the sensors to an electrical signal.

6. An endoscope system according to claim 1, wherein the sensors comprise a coil provided on one of the two optical substrates and a metal plate provided on the other and detect the distance between the two optical substrates by detecting the impedance of the coil.

7. An endoscope system according to claim 1, wherein the securing portion secures the electrical wires to the optical substrates.

8. An endoscope system according to claim 7, wherein the securing portion secures the electrical wires to the optical substrate at a fixed side.

9. An endoscope system according to claim 1, wherein the securing portion secures the electrical wires to a base member to which the fixed-side optical substrate is fixed.

10. An endoscope system according to claim 1, wherein the securing portion secures the electrical wires to a terminal block that relays the electrical wires from the sensors to the electrical circuit.

11. An endoscope system according to claim 1, wherein the securing portion secures the electrical wires with an adhesive.

12. An endoscope system according to claim 1, wherein the securing portion secures a plurality of the electrical wires from the sensors to each other.

13. An endoscope system according to claim 12, wherein the securing portion secures the electrical wires from the sensors provided on different optical substrates to each other.

14. An endoscope system according to claim 12, wherein the securing portion secures the electrical wires from the sensors provided on the same optical substrate to each other.

15. An endoscope system according to claim 1, wherein a variable spectral device comprising the optical substrate, the actuator, and the sensors is disposed at a distal end of the insertion portion.

16. An endoscope according to claim 1, wherein a variable spectral device comprising the optical substrate, the actuator, and the sensors is disposed at a base end at the opposite end from the distal end of the insertion portion.
